(19) United States
(12) Patent Application Publication
(10) Pub. No.: US 2023/0009533 A1
(43) Pub. Date: Jan. 12, 2023

(54) MOVEABLE IONIZATION UNIT FOR CLEANING AIR IN A ROOM

(71) Applicant: PHILIP MORRIS PRODUCTS S.A., Neuchatel (CH)

(72) Inventors: Rui Nuno Batista, Morges (CH); Ricardo Cali, Mannheim (DE); Louis Beck, Lausanne (CH)

(21) Appl. No.: 17/911,852

(22) PCT Filed: Mar. 16, 2021
(86) PCT No.: PCT/EP2021/056634

§ 371 Date: Sep. 15, 2022

(30) Foreign Application Priority Data
Mar. 16, 2020 (EP) 20163331.0

(51) Int. Cl. A61L 9/22; A47L 9/28

(52) U.S. Cl. CPC A61L 9/22; A47L 9/2852; A47L 2201/04; A61L 2209/111

(57) ABSTRACT

An Ionization unit (7) for cleaning air in a room (1) with a ceiling (3) and a floor (5), comprising an ionizer (19) configured to electrically charge particles in the air; and a support unit (13) configured to enable the ionization unit (7) to be positioned at a distance of at least 50 centimeters above the floor (5) of the room (1), wherein the support unit (13) allows the ionization unit (7) to travel within the room (1) while being distanced from the floor (5).

The present disclosure relates to cleaning air in a room.

Ionic air purifiers for home use are known from practice. Those devices comprise stationary ionizers creating negative ions in the air.

DE 10 2004 036 459 A1 discloses a self-driving disc-shaped cleaning robot having a vacuum unit for sucking in dust from a floor. The cleaning robot further comprises a negative ion generation unit provided in the robot body to carry out an air cleaning operation, either at the same time as carrying out a vacuum cleaning operation or selectively.

According to an aspect of the present invention, there is provided an ionization unit for cleaning air in a room with a ceiling and a floor. The ionization unit comprises an ionizer and a support unit. The ionizer is configured to electrically charge particles in the air. The support unit is configured to enable the ionization unit to be positioned at a distance of at least 50 centimeters above the floor of the room. The support unit allows the ionization unit to travel within the room while being distanced from the floor.

When the ionizer electrically charges particles in the air, those particles tend to form clusters with other particles in the air due to electrostatic interaction. Clusters of particles in the air may descend within the room at a higher rate than individual particles. Clusters of particles may descend within the room at a higher velocity than individual particles. Electrically charging particles in the air at a distance above the floor of the room increases the rate at which particles in the air settle down within the room. Particles that settle down no longer float in the air. As the support unit may travel within the room while being distanced from the floor (at a distance of at least 50 centimeters above the floor), different regions of the room may be treated at different times. The ionization unit may move through the room to successively carry out air cleaning operations at different locations within the room. The ionization unit may move to specific locations within the room where air cleaning is currently needed.

The distance of the ionization unit above the floor of the room may be measured in a vertical direction. The distance of the ionization unit above the floor of the room may be the distance between the floor of the room and the ionization unit measured in a vertical direction at a point of the ionization unit at which the distance between the ionization unit and the floor is the lowest.

The support unit may be configured to enable the ionization unit to be positioned at a distance of at least 80 centimeters, or at least 100 centimeters, or at least 150 centimeters, or at least 200 centimeters, or at least 250 centimeters above the floor. If the ionization unit is positioned high above the floor, particles that are electrically charged by the ionizer have ample opportunity to form clusters with other particles due to electrostatic interaction on their way down.

The ionization unit may be positioned at a distance of no more than 10 meters, or no more than 8 meters, or no more than 7 meters, or no more than 6 meters, or no more than 5 meters, or no more than 4 meters, or no more than 3 meters above the floor.

The distance between the ionization unit and the floor may be adjustable. Adjusting the distance between the ionization unit and the floor may allow operating the ionization unit under optimal conditions under different circumstances. Further, adjusting the distance may facilitate moving the ionization unit through the room. Adjusting the distance between the ionization unit and the floor may enable the ionization unit to traverse obstacles. For example, the distance between the floor and the ionization unit may be reduced to enable the ionization unit to pass through a door.

The ionization unit may comprise a drive unit configured to move the ionization unit through the room while being distanced from the floor.

The support unit may be configured to suspend the ionization unit from the ceiling of the room. The support unit may enable the ionization unit to hang from the ceiling of the room. The ceiling of the room may be used as a support structure supporting the ionization unit. Suspending the ionization unit from the ceiling of the room may allow the ionization unit to operate within the room without disturbing activities carried out below the ionization unit. For example, people may pass below the ionization unit. The ionization unit may be movable at the ceiling of the room while being suspended from the ceiling of the room.

The support unit may comprise suction cups configured to hold the ionization unit at the ceiling of the room. Proving suction cups at the ionization unit may allow holding the ionization unit at the ceiling of the room without having to specifically modify the ceiling of the room. The support unit may comprise a suction device for selectively applying an underpressure between the suction cups and the ceiling of the room.

The suction cups may be provided at a rotatable structure of the ionization unit for moving the ionization unit through the room while being distanced from the floor. The rotatable structure may be part of a drive unit of the ionization unit. The rotatable structure may, for example, comprise one or more wheels or a caterpillar device.

The support unit may comprise a rail drive for engaging rails provided at the ceiling of the room.

The support unit may comprise an airborne unit configured to enable the ionization unit to fly or float above the floor. If the ionization unit flies or floats above the floor, there is no need for the ionization unit to engage with any support structure. When the ionization unit flies or floats above the floor, a distance between the ionization unit and the floor may be easily adjustable. The distance between the ionization unit and the floor may be freely adjustable. The ionizer may be provided at the airborne unit.

The airborne unit may comprise a lifting cell for receiving gas having a lower density than air. A lifting cell may enable the ionization unit to fly or float above the floor in a relatively simple and cost-effective manner.

The ionization unit may be pulled by a unit moving on the floor to move within the room. If the ionization unit is pulled, the ionization unit does not need to have its own drive unit.

The ionization unit may comprise a drive unit, such as a propeller, to move within the room.

The airborne unit may comprise one or more propellers to enable the ionization unit to fly or float above the floor. In particular, the airborne unit could comprise a drone. The ionizer could be fixed to the drone.

The ionizer may be configured to charge the particles in the air by way of a corona discharge.

The ionizer may comprise a piezoelectric transformer. The piezoelectric transformer may be a Rosen-type piezoelectric transformer. The piezoelectric transformer may use a Piezoelectric Direct Discharge Effect to generate ions. The piezoelectric transformer may generate cold plasma. A resonance frequency of the piezoelectric transformer may be between 10 kHz and 500 kHz, preferably between 200 kHz and 300 kHz.

According to another aspect of the present invention, there is provided a system for cleaning air in a room with a ceiling and floor. The system comprises an ionization unit and a cleaning robot. The ionization unit may correspond to the ionization unit as described above. The cleaning robot is configured to move on the floor of the room. The ionization unit and the cleaning robot are configured to move through the room in a coordinated manner.

As the ionization unit and the cleaning robot move through the room in a coordinated manner, the ionization unit and the cleaning robot may efficiently work together to clean a room.

The ionization unit and the cleaning robot may be configured to move so as to be positioned above each other. The ionization unit and the cleaning robot may be configured to move so as to at least partially overlap along a vertical direction. If the ionization unit and the cleaning robot overlap in a vertical direction, there may be at least one point on the ionization unit that lies directly above a point on the cleaning robot with respect to a vertical direction. The ionization unit and the cleaning robot may be configured to move so as to be positioned above each other within a certain tolerance. For example, the cleaning robot may move so as to remain within a certain region around a vertical projection of the ionization unit onto the floor of the room.

One of the ionization unit and the cleaning robot may be configured as lead unit and the other one of the ionization unit and the cleaning unit may be configured as follow unit. The follow unit may move according to movements of the lead unit. A movement pattern of the follow unit may be determined based on movements of the lead unit. There may be a time delay between movement of the lead unit and movement of the follow unit. The time delay may be at least 1 second, or at least 3 seconds, or at least 5 seconds, or at least 20 seconds, or at least 40 seconds, or at least 60 seconds, or at least 120 seconds. The time delay may be lower than 500 seconds, or lower than 300 seconds, or lower than 150 seconds, or lower than 80 seconds, or lower than 30 seconds, or lower than 10 seconds. The time delay may be determined based on properties of clusters of particles descending from the ionization unit. The cleaning robot may be configured to follow a movement of the ionization unit. The ionization unit may be configured to follow a movement of the cleaning robot.

The ionization unit and the cleaning robot may be configured to exchange data with each other. The ionization unit and the cleaning robot may directly communicate with each other. Alternatively or in addition, the ionization unit and the cleaning robot may communicate with each other via an external unit, such as an external control unit. Data exchanged between the ionization unit and the cleaning robot may comprise positon information indicating the current position of the ionization unit or the cleaning robot. The data exchanged between the ionization unit and the cleaning robot may comprise guidance information for guiding the movement of at least one of the ionization unit and the cleaning robot within the room.

Communication between the ionization unit and the cleaning robot may comprise wireless communication. Alternatively or in addition, the ionization unit and the cleaning robot may communicate via a wired connection.

The ionization unit and the cleaning robot may be connected by a connection member. The connection member may be configured to transfer force between the cleaning robot and the ionization unit. The ionization unit may be pulled through the room by the cleaning robot via the connection member. The ionization unit may be pushed through the room by the cleaning robot via the connection member. If the cleaning robot pulls the ionization unit via the connection member, coordination of movement between the cleaning robot and the ionization unit can be achieved without a complicated control structure. Further, there may be no need for the ionization unit to have a drive unit for propelling the ionization unit on its own.

The connection member may be flexible. The connection member may be non-supportive. If the connection member is non-supportive, the connection member may be insufficient to hold the ionization unit at its position above the floor of the room on its own. For example, the connection member may comprise a rope, or a cord, or a wire, or a rod.

The connection member may comprise a wire for exchanging data between the ionization unit and the cleaning robot.

The cleaning robot may comprise an electrically charged surface configured to attract charged particles. The electrically charged surface may attract particles electrically charged by operation of the ionizer of the ionization unit. The electrically charged surface of the cleaning robot may serve to guide particles charged by the ionization unit towards the cleaning robot. The cleaning robot may be configured to collect particles that are electrically charged by the ionization unit.

The cleaning robot may comprise a vacuum unit. The vacuum unit may comprise a filter unit configured to filter dust from air and an airflow unit configured to suck in air from the room and provide the air to the filter unit. The cleaning robot may be configured to remove dust from the air by filtration. In particular, individual particles or clusters of particles falling down due to gravity may be removed from the air by the cleaning robot.

The airflow unit may be configured to suck in the air from the room through an inlet opening of the cleaning robot. The inlet opening may face upwards. If the inlet opening faces upwards, individual particles or clusters of particles falling down due to gravity are more likely to be sucked in through the inlet opening.

Optionally, the cleaning robot may comprise one or more additional inlet openings facing towards the floor. The cleaning robot may be configured to suck in air through the additional cleaning opening facing towards the floor to provide a function of vacuum cleaning the floor.

According to another aspect of the present invention, there is provided a method for cleaning air in a room with a ceiling and floor. The method comprises moving an ionization unit at a distance of at least 50 centimeters above the floor of the room. The method further comprises electrically charging particles in the air by the ionization unit.

The ionization unit may charge the particles in the air by way of corona discharge.

The method may further comprise moving a cleaning robot at the floor of the room in coordination with the movement of the ionization unit.

The ionization unit and the cleaning robot may move in a coordinated manner so as to be position above each other.

The ionization unit may be displaced by the cleaning robot. In particular, the ionization unit may be pulled by the cleaning robot.

The cleaning robot may attract charged particles in the air with at least one electrically charged surface.

The cleaning robot may suck in air from within the room. The cleaning robot may filter the air sucked in from within the room.

The cleaning robot may suck in the air through an inlet opening facing towards an upside direction.

The method may comprise adjusting the distance between the ionization unit and the floor.

The ionization unit may move at the ceiling of the room.

The ionization unit may hang from the ceiling while moving at the ceiling.

The ionization unit may be held at the ceiling with suction cups provided at the ionization unit.

The suctions may be provided at a rotatable structure of the ionization unit for moving the ionization unit.

The ionization unit may fly or float in the room.

The particles may comprise dust particles or other particulate matter. The particles may comprise particles floating in the air.

According to another aspect of the present invention, there is provided a use of an ionization unit moving within a room to accelerate gravitation-based descent of particles within the room. The particles may comprise dust particles or other particulate matter. The particles may comprise particles floating in the air.

The ionization unit may increase the rate at which particles settle down in the room. By causing particles to settle down at an increased rate, the particles may be removed from the air within the room. The particles may be made accessible for being cleaned by a cleaning operation carried out at the floor of the room. As the ionization unit moves within the room, the effect of accelerating gravitation-based descent of particles is not limited to a particular location within the room.

The ionization unit may move at a vertical distance of at least 50 centimeters, or at least 80 centimeters, or at least 100 centimeters, or at least 150 centimeters, or at least 200 centimeters, or at least 250 centimeters above the floor of the room.

The ionization unit may electrically charge the particles in the air to cause the particles to form clusters.

The ionization unit may fly or float in the room.

Different aspects of the present invention provide an ionization unit, a system, a method, and a use. Any one or more of the features of these aspects may be combined with any one or more features of all other aspects.

Below, there is provided a non-exhaustive list of non-limiting examples, embodiments or aspects of the invention. Any one or more of the features of these examples, embodiments or aspects of the invention may be combined with any one or more features of another example, embodiment or aspect described herein.

Example A1: Ionization unit for cleaning air in a room with a ceiling and a floor, comprising:
an ionizer configured to electrically charge particles in the air; and
a support unit configured to enable the ionization unit to be positioned at a distance of at least 50 centimeters above the floor of the room, wherein the support unit allows the ionization unit to travel within the room while being distanced from the floor.

Example A2: Ionization unit according to example A1, wherein the support unit is configured to enable the ionization unit to be positioned at a distance of at least 80 centimeters or at least 100 centimeters or at least 150 centimeters or at least 200 centimeters or at least 250 centimeters above the floor.

Example A3: Ionization unit according to example A1 or A2, further comprising a drive unit configured to move the ionization unit through the room while being distanced from the floor.

Example A4: Ionization unit according to any one of examples A1 to A3, wherein the support unit is configured to suspend the ionization unit from the ceiling of the room.

Example A5: Ionization unit according to any one of any one of examples A1 to A4, wherein support unit comprises suction cups configured to hold the ionization unit at the ceiling of the room.

Example A6: Ionization unit according to example A5, wherein the suction cups are provided at a rotatable structure of the ionization unit for moving the ionization unit through the room while being distanced from the floor.

Example A7: Ionization unit according to any one of examples A1 to A3, wherein the support unit comprises an airborne unit configured to enable the ionization unit to fly or float above the floor.

Example A8: Ionization unit according to example A7, wherein the airborne unit comprises a lifting cell for receiving gas having a lower density than air.

Example A9: Ionization unit according to any one of any one of examples A1 to A8, wherein the ionizer is configured to charge the particles in the air by way of corona discharge.

Example A10: Ionization unit according to any one of any one of examples A1 to A9, wherein the ionizer comprises a piezoelectric transformer.

Example B1: System for cleaning air in a room with a ceiling and a floor, comprising:
an ionization unit, in particular according to any one of any one of examples A1 to A10; and
a cleaning robot configured to move on the floor,
wherein the ionization unit and the cleaning robot are configured to move through the room in a coordinated manner.

Example B2: System according to example B1, wherein the ionization unit and the cleaning robot are configured to move so as to be positioned above each other.

Example B3: System according to example B1 or B2, wherein the ionization unit and the cleaning robot are configured to exchange data with each other.

Example B4: System according to any one of examples B1 to B3, wherein the ionization unit and the cleaning robot are connected by a connection member.

Example B5: System according to example B4, wherein the connection member is flexible.

Example B6: System according to example B4 or B5, wherein the connection member comprises a wire for exchanging data between the ionization unit and the cleaning robot.

Example B7: System according to any one of examples B1 to B6, wherein the cleaning robot comprises an electrically charged surface configured to attract charged particles.

Example B8: System according to any one of examples B1 to B7, wherein the cleaning robot comprises a vacuum unit, the vacuum unit comprising a filter unit configured to filter dust from air and an air flow unit configured to suck in air from the room and provide the air to the filter unit.

Example B9: System according to example B8, wherein the air flow unit is configured to suck in the air from the room through an inlet opening of the cleaning robot, wherein the inlet opening faces upwards.

Example C1: Method for cleaning air in a room with a ceiling and a floor, comprising:
moving an ionization unit at a distance of at least 50 centimeters above the floor of the room; and
electrically charging particles in the air by the ionization unit.

Example C2: Method according to example C1, wherein the ionization unit charges the particles in the air by way of corona discharge.

Example C3: Method according to example C1 or C2, further comprising moving a cleaning robot at the floor of the room in coordination with the movement of the ionization unit.

Example C4: Method according to example C3, wherein the ionization unit and the cleaning robot move in a coordinated manner so as to be positioned above each other.

Example C5: Method according to example C3 or C4, wherein the ionization unit is displaced by the cleaning robot.

Example C6: Method according to any one of examples C3 to C5, wherein the cleaning robot attracts charged particles in the air with at least one electrically charged surface.

Example C7: Method according to any one of examples C3 to C6, wherein the cleaning robot sucks in air from within the room and filters the air.

Example C8: Method according to example C7, wherein the cleaning robot sucks in the air through an inlet opening facing towards an upside direction.

Example C9: Method according to any one of examples C1 to C8, wherein the ionization unit moves at the ceiling of the room.

Example C10: Method according to any one of examples C1 to C9, wherein the ionization unit hangs from the ceiling while moving at the ceiling.

Example C11: Method according to any one of examples C1 to C10, wherein the ionization unit is held at the ceiling with suction cups provided at the ionization unit.

Example C12: Method according to example C11, wherein the suction cups are provided at a rotatable structure of the ionization unit for moving the ionization unit.

Example C13: Method according to any one of examples C1 to C12, wherein the ionization unit flies or floats in the room.

Example D1: Use of an ionization unit moving within a room to accelerate gravitation-based descent of particles within the room.

Example D2: Use according to example D1, wherein the ionization unit moves at a vertical distance of at least 50 centimeters or at least 80 centimeters or at least 100 centimeters or at least 150 centimeters or at least 200 centimeters or at least 250 centimeters above a floor of the room.

Example D3: Use according to example D1 or D2, wherein the ionization unit electrically charges the particles in the air to cause the particles to form clusters.

Example D4: Use according to any one of examples D1 to D3, wherein the ionization unit flies or floats in the room.

Examples with now be further described with reference to the figures in which:

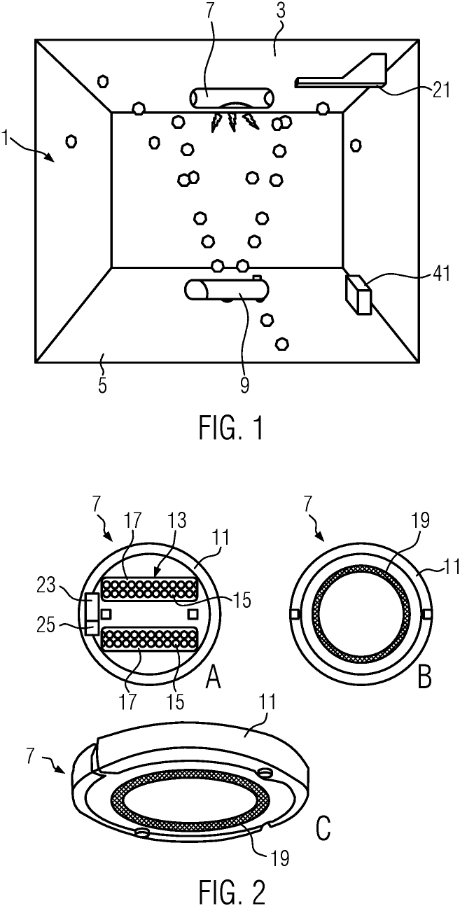

FIG. 1 schematically shows a system comprising an ionization unit and a cleaning robot within a room according to an embodiment of the invention;

FIG. 2 shows schematic top, bottom and perspective views of the ionization unit of FIG. 1;

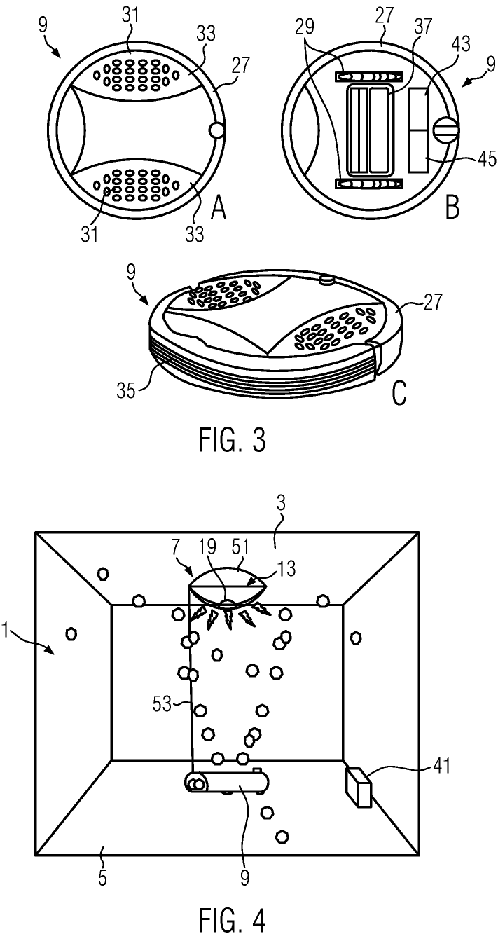

FIG. 3 shows schematic top, bottom and perspective views of the cleaning robot of FIG. 1;

FIG. 4 schematically shows a system comprising an ionization unit and a cleaning robot in a room according to another embodiment of the invention; and

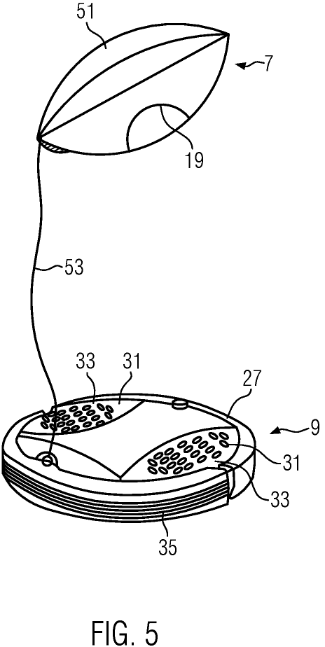

FIG. 5 schematically shows a perspective view of the ionization unit and the cleaning robot of FIG. 4.

FIG. 1 shows a system for cleaning air in a room 1 according to an embodiment. The room comprises a ceiling 3 and a floor 5. The system comprises an ionization unit 7 and a cleaning robot 9.

In the embodiment of FIG. 1, the ionization unit 7 moves at the ceiling 3 of the room 1. The ionization unit 7 hangs from the ceiling 3 of the room 1.

FIG. 2 shows details of the ionization unit of FIG. 1. Part A of FIG. 2 shows a top view of the ionization unit 7 of FIG. 1, Part B of FIG. 2 shows a bottom view of the ionization unit 7 of FIG. 1, and Part C of FIG. 2 shows a perspective bottom view of the ionization unit 7 of FIG. 1.

The ionization unit 7 comprises a main body 11 and a support unit 13 configured to hold the ionization unit 7 at the ceiling 3 of the room 1. The support unit 13 comprises suction cups 15 for engaging the ceiling 3 of the room 1. The ionization unit 7 comprises a suction unit configured to create an underpressure between the suction cups 15 and a lower surface of the ceiling 3 to hold the ionization unit 7 at the lower surface of the ceiling 3. The suction cups 15 are provided at a rotatable structure 17 of the ionization unit 7. In the present embodiment, the rotatable structure 17 comprises two caterpillar devices. Alternatively, the rotatable structure 17 could comprise one or more wheels, for example. The ionization unit 7 comprises a drive unit for driving the rotatable structure 17 to move the ionization unit 7 along the lower surface of the ceiling 3.

The ionization unit 7 comprises an ionizer 19. The ionizer 19 may be provided at a lower surface of the ionization unit 7. The ionizer 19 may comprise a piezoelectric transformer, in particular a Rosen-type piezoelectric transformer. The ionizer 19 is configured to electrically charge particles in the air, such as dust particles in the air. The ionizer 19 may charge the particles in the air by way of a corona discharge. If particles in the air are electrically charged by the ionizer 19, the particles tend to form clusters with other particles in the air due to electrostatic interaction. Such clusters of particles have a higher tendency to settle down within the room 1 due to gravity than individual particles. Clusters of particles may descend within the room 1 at a higher velocity than individual particles.

The ionization unit 7 may comprise a rechargeable power source, such as a rechargeable battery. The ionization unit 7 may return to a base station 21 from time to time to recharge the rechargeable power source. The base station 21 may be provided at the ceiling 3. Alternatively, the ionization unit 7 could be removed from the ceiling 3 after use to be recharged. As a further alternative, the ionization unit 7 could be connected to a power source by wire.

The ionization unit 7 comprises a control unit 23. The control unit 23 may control the drive unit and the ionizer 19 of the ionization unit 7. The ionization unit 7 may comprise a sensor unit 25. The sensor unit 25 may, for example, comprise an obstacle sensor. The sensor unit 25 may, for example, comprise a particle sensor.

As mentioned, the system further comprises a cleaning robot 9. The cleaning robot 9 moves on the floor 5 of the room 1. Details of the cleaning robot 9 of FIG. 1 are shown in FIG. 3. Part A of FIG. 3 shows a top view of the cleaning robot 9, Part B of FIG. 3 shows a bottom view of the cleaning robot 9 and Part C of FIG. 3 shows a top perspective view of the cleaning robot 9.

The cleaning robot 9 comprises a main body 27. The cleaning robot 9 may be self-driving. The cleaning robot 9 comprises a drive assembly 29 enabling the cleaning robot 9 to move on the floor 5. The cleaning robot 9 comprises a vacuum unit. The vacuum unit has a filter unit provided within the main body 27 and configured to filter dust from air. Further, the vacuum unit comprises an airflow unit configured to suck in air from the room 1 and provide the air to the filter unit. The cleaning robot 9 comprises an inlet opening 31 through which the vacuum unit sucks in air. The inlet opening 31 faces away from the floor 5 and is open towards an upper side. As the inlet opening 31 faces upward, particles or clusters of particles falling down by gravity may be directly sucked in through the inlet opening 31. The cleaning robot 9 comprises an electrically charged surface 33 provided at the inlet opening 31. In the present embodiment, the electrically charged surface 33 is plate-shaped with through holes for allowing air and dust to pass through. The electrically surface 31 may attract dust particles. In particular, the electrically charged surfaced 33 may attract particles that were electrically charged by the ionization unit 7. Air sucked in through the inlet opening 31 may be filtered within the main body 27 of the cleaning robot 9 and may then be discharged through an outlet opening 35 provided at a lateral side of the main body 27.

In addition or as an alternative to the inlet opening 31 facing upwards, the cleaning robot 9 may comprise an inlet opening 37 facing towards the floor 5. The vacuum unit may suck in air through the inlet opening 37 facing towards the floor 5 to provide a floor cleaning function similar to the floor cleaning function of standard cleaning robots. Air sucked in through the inlet opening 37 facing the floor 5 may be discharged through the outlet opening 35 after being filtered.

The cleaning robot 9 may comprise a power source. The power source of the cleaning robot 9 may be a rechargeable power source, such as a rechargeable battery. The cleaning robot 9 may return to a base station 41 from time to time to be recharged. Alternatively, the cleaning robot 9 could be powered via a wired connection.

The cleaning robot 9 comprises a control unit 43 controlling the drive unit 29 to move the cleaning robot 9 on top of the floor 5. The control unit 43 may also control other functions of the cleaning robot 9. The cleaning robot 9 may comprise a sensor unit 45. The sensor unit 45 may comprise an obstacle sensor. The sensor unit 45 may comprise a particle sensor.

The ionization unit 7 and the cleaning robot 9 may move through the room 1 in a coordinated manner. The movement of the ionization unit 7 within the room 1 may be coordinated with the movement of the cleaning robot 9 on the floor 5. One of the ionization unit 7 and the cleaning robot 9 may be configured as a lead device. The other one of the ionization unit 7 and the cleaning robot 9 may be configured as a follow device. The follow device may be configured to move based on the movement of the lead device. The follow device may be configured to follow the lead device through the room 1. The lead device may be configured to move through the room 1 based on a predetermined movement pattern. The predetermined movement pattern may be programmed by a user or may be derived based on previous runs. The lead unit may move through the room 1 based on a random walk scheme. The lead unit may move through the room 1 based on output of the sensor unit 25, 45 of the lead unit. For example, the lead unit may be configured to move through the room 1 based on output of an obstacle sensor of the lead unit, possibly in combination with a random walk scheme. The lead unit may be configured to move through the room based on output of a particle sensor of the lead unit. For example, the lead unit may remain stationary as long as its particle sensor detects a particle density in the air that is above a predetermined threshold at the location of the lead unit. Once the particle density detected by the particle sensor of the lead unit falls below the threshold value, the lead unit may move to another location within the room 1.

The ionization unit 7 and the cleaning robot 9 may be configured to move so as to be positioned above each other. The ionization unit 7 and the cleaning robot 9 may be configured to move so as to at least partially overlap along a vertical direction. The ionization unit 7 and the cleaning robot 9 may be configured to move so as to be positioned above each other within a certain tolerance. For example, the cleaning robot 9 may move so as to remain within a certain region around a vertical projection of the ionization unit 7 onto the floor 5 of the room 1.

The ionization unit 7 and the cleaning robot 9 may be in data communication with each other. Preferably, the ionization unit 7 and the cleaning robot 9 communicate wirelessly. For example, the lead unit may communicate movement information to the follow unit to allow the follow unit to move in coordination with the lead unit.

There may be direct data communication between the ionization unit 7 and the cleaning robot 9. It would also be conceivable that both the ionization unit 7 and the cleaning robot 9 are in communication with an external entity, such as an external control unit. Such external control unit might, for example, be provided in the base station 21 of the ionization unit 7 or in the base station 41 of the cleaning robot 9, or could be part of a smart house control unit. The external control unit could coordinate movement of the ionization unit 7 and the cleaning robot 9.

FIG. 1 shows the ionization unit 7 moving at the ceiling 3 of the room 1. As an alternative, the ionization unit 7 could move at another support structure provided at a distance to the floor 5. For example, the ionization unit 7 could move at an intermediate ceiling hanging from the ceiling 3. The ionization unit 7 could also move at walls of the room 1.

FIG. 4 shows another embodiment of a system for cleaning air in a room 1 with a ceiling 3 and floor 5. The system again comprises an ionization unit 7 and a cleaning robot 9. The cleaning robot 9 may be the same as the cleaning robot 9 described with respect to FIGS. 1 to 3. The ionization unit 7 of the embodiment show in FIG. 4 is different from the ionization unit 7 shown in FIGS. 1 and 2.

According to the embodiment of FIG. 4 the ionization unit 7 comprises a lifting cell 51 filled with gas having a lower density than air. For example, the lifting cell 51 may be filled with helium. The lifting cell 51 serves as a support unit 13 allowing the ionization unit 7 to move above the floor 5 of the room 1. The ionization unit 7 moves at least 50 centimeters above the floor 5. The ionization unit 7 may move at a distance of at least 80 centimeters, or a distance of at least 100 centimeters, or at a distance of at least 150 centimeters, or at a distance of at least 200 centimeters, or at a distance of at least 250 centimeters above the floor 5.

In the illustrated embodiment, the ionization unit 7 is connected to the cleaning robot 9 with a connection member 53. The connection member 53 is non-supportive. This means that if the helium would be released from within the lifting cell 51, the lifting cell 51 would not be held at it position by the connection member 53, but would fall down due to gravity. The connection member 53 may be flexible. The connection member 53 may comprise a rope, or a cord, or a wire, or a rod for example.

An ionizer 19 may be provided at the lifting cell 51. The ionizer 19 may be configured in the same way as the ionizer 19 described with respect to FIGS. 1 to 3. The ionizer 19 is operable to electrically charge particles in the air.

According to the embodiment of FIG. 4, the cleaning robot 9 on the floor 5 acts as the lead unit. The lifting cell 51 is moved according to the movement of the cleaning robot 9, when the cleaning robot 9 moves on the floor 5. The ionization unit 7 is pulled along with the cleaning robot 9 via the connection member 53. Due to the ionization unit 7 being pulled along by the cleaning robot 9, the ionization unit 7 does not require separate drive means. Further, no complex means for coordinating movement of the ionization unit 7 with the movement of cleaning robot 9 are required.

The connection member 53 may comprise a conductive wire for supplying the ionization unit 7 with power. In particular, the ionizer 19 may be provided with power through the connection member 53. Alternatively, the ionization unit 7 may comprise its own power source, such as a rechargeable battery.

According to an embodiment, a distance between the ionization unit 7 and the floor 5 is adjustable via the connection member 53. For example, the cleaning robot 9 may be configured to pull in the connection member 53 to lower the ionization unit 7 or to release additional length of the connection member 53 to increase the distance between the ionization unit 7 and the floor 5.

FIG. 5 shows a schematic view of the cleaning robot 9 with the ionization unit 7 having the lifting cell 51 connected to the cleaning robot 9 by the connection member 53.

According to the embodiment of FIGS. 4 and 5, the ionization unit 7 is not provided with a separate drive unit. However, it would be conceivable to not provide the connection member 53 and instead provide the ionization unit 7 having the lifting cell 51 with separate drive means, such as propeller drive means. In this case, the movements of the ionization unit 7 and the cleaning robot 9 could be coordinated in a similar manner as described for the embodiment of FIG. 1.

For the purpose of the present description and of the appended claims, except where otherwise indicated, all numbers expressing amounts, quantities, percentages, and so forth, are to be understood as being modified in all instances by the term “about”. Also, all ranges include the maximum and minimum points disclosed and include any intermediate ranges therein, which may or may not be specifically enumerated herein. In this context, therefore, a number A is understood as A ±10% of A.

1. An ionization unit for cleaning air in a room with a ceiling and a floor, comprising:
an ionizer configured to electrically charge particles in the air; and
a support unit configured to enable the ionization unit to be positioned at a distance of at least 50 centimeters above the floor of the room, wherein the support unit allows the ionization unit to travel within the room while being distanced from the floor.

2. The ionization unit according to claim 1, wherein the support unit is configured to suspend the ionization unit from the ceiling of the room.

3. The ionization according to claim 1, wherein support unit comprises suction cups configured to hold the ionization unit at the ceiling of the room.

4. The ionization unit according to claim 1, wherein the support unit comprises an airborne unit configured to enable the ionization unit to fly or float above the floor.

5. A system for cleaning air in a room with a ceiling and a floor, comprising:
an ionization unit, in particular according to any one of the preceding claims; and
a cleaning robot configured to move on the floor,
wherein the ionization unit and the cleaning robot are configured to move through the room in a coordinated manner.

6. The system according to claim 5, wherein the ionization unit and the cleaning robot are connected by a connection member.

7. The system according to claim 5, wherein the cleaning robot comprises a vacuum unit with an air flow unit configured to suck in air from the room through an inlet opening of the cleaning robot, wherein the inlet opening faces upwards.

8. A method for cleaning air in a room with a ceiling and a floor, comprising:
moving an ionization unit at a distance of at least 50 centimeters above the floor of the room; and
electrically charging particles in the air by the ionization unit.

9. The method according to claim 8, further comprising moving a cleaning robot at the floor of the room in coordination with the movement of the ionization unit.

10. The method according to claim 9, wherein the ionization unit is displaced by the cleaning robot.

11. The method according to claim 9, wherein the cleaning robot sucks in air from within the room through an inlet opening facing towards an upside direction.

12. The method according to claim 8, wherein the ionization unit moves at the ceiling of the room.

13. The method according to claim 8, wherein the ionization unit hangs from the ceiling while moving at the ceiling.

14. The method according to claim 8, wherein the ionization unit flies or floats in the room.

15. Use of an ionization unit moving within a room at a distance of at least 50 centimeters above a floor of the room to accelerate gravitation-based descent of particles within the room.
